(19) United States
(12) Patent Application Publication
(10) Pub. No.: US 2002/0120201 A1
(43) Pub. Date: Aug. 29, 2002

(54) Hemodynamic analysis device and method

(76) Inventors: Shiu-Shin Chio, Rancho Santa Fe, CA (US); Sen Lin-Liu, San Diego, CA (US); Jeffrey Tsai, Fresno, CA (US)

Correspondence Address: E. Victor Indiano, Esq., INDIANO & VAUGHAN, PA, Suite 850, One North Pennsylvania Street, Indianapolis, IN, 46204, US

(21) Appl. No.: 10/080,764

(22) Filed: Feb. 22, 2002

Related U.S. Application Data
(60) Provisional application No. 60/271,235, filed on Feb. 23, 2001.

(51) Int. Cl. A61B 5/02

(52) U.S. Cl. 600/490

(57) ABSTRACT

A method for remotely monitoring the cardiovascular condition of a patent includes using a data acquisition device at a patient site to non-invasively acquire cardiovascular condition information from a patient. The cardiovascular condition information includes a data stream of pulse pressure-related data. The cardiovascular condition information acquired by the data acquisition device is transmitted to a remote processor capable of performing data processing and data storage functions on the transmitted cardiovascular condition information. The cardiovascular condition information processed by the remote processor is transmitted to a data display device at a healthcare provider site remote from the data acquisition device for permitting the healthcare provider to use the cardiovascular condition information to monitor the cardiovascular condition of the patient.

### I. TECHNICAL FIELD OF THE INVENTION

The present invention generally relates to the use of a health condition monitoring device, in conjunction with a global computer network, such as the Internet, to monitor the condition of the health of a patient while the patient is in or out of a healthcare facility, or for that matter, at any place where a health monitoring device exists that is capable of recording patient health data, and a wired or wireless communications device exists that is capable of transmitting and receiving data over the Internet.

### II. BACKGROUND OF THE INVENTION

A. Overview

The present invention broadly relates to the application of server intelligence to the management, interpretation and use of health data received over the a global computer network such as the Internet. The system disclosed herein can therefore be referred to as an “intelligent Internet health information management” system that addresses the fundamental principles of general application of monitoring devices to the emerging art of intelligent Internet health information management.

The present invention more specifically relates to the use of a software system that employs health monitoring devices and the Internet to collect, analyze and distribute data and to help a physician provide frequent-interval, long-term monitoring of patients.

B. Background

Heart failure is a clinical syndrome that has become more prevalent in recent years.¹ Almost five million Americans are afflicted with congestive heart failure (CHF), and each year Approximately 400,000 new cases of CHF are diagnosed. Disease prevalence is expected to reach 10 million cases in the U.S. alone by the year 2007.² Hospitalization costs account for a major portion of expense related to the management of heart failure patients. ¹J. B. O'Connell, M.D., The Economic Burden of Heart Failure. Clin. Cardol. Vol. 23 (Suppl. III.), III-6-III-10 (2000) ²Rich MW: Epidemiology, Pathophysiology, and Etiology of Congestive Heart Failure in Older Adults. J. Am Geriatr Soc 1997; 45: 968-974

One of the problems facing healthcare today, and especially cardiovascular care, relates to the issue of monitoring health-related parameters for patients, and in particular monitoring a patient's cardiovascular condition and cardiovascular parameters. Many of the parameters that one would desire to monitor are best monitored currently in a doctor's office or healthcare institution setting where tests such as blood pressure and electrocardiograms can be administered to monitor a patient's health parameters. Unfortunately, monitoring a patient's hemodynamic parameters becomes very inconvenient for the patient because he/she must travel to and from the medical facility (e.g. doctor's office, clinic) each time monitoring occurs. These frequent trips present a hardship to the patient, especially if daily tests (and hence, daily trips) are required.

Despite significant advances in medical technology, one of the greatest challenges in managing patient care springs from the need for readily accessible, objective data that permits the healthcare provider to determine disease progression and/or treatment effectiveness.³ As such, obtaining and trending this data depends upon technology that produces valid, reproducible, and cost effective measurements of cardiac function in a timely manner. Although both invasive and noninvasive technologies have been developed and used effectively in the assessment, diagnosis, and evaluation of treatment outcomes, most require specialized environments, costly equipment, and specially trained medical personnel to obtain and/or interpret data. Because of the cost and/or risk associated with these technologies, repeated hemodynamic measurements that would enhance medical management and permit healthcare providers to fine tune a patient's care are usually not obtained in an ambulatory care setting.⁴ ³Buell JC. A Practical, Cost Effective, Non-Invasive System for Cardiac Output and Hemodynamic Analysis. Am Heart J. 1988: 116:657-664 ⁴B. Greenberg, M.D., “Reproducibility of Impedance Cardiography Hemodynamic Measures in Clinically Stable Heart Failure Patients.” Congestive Heart Failure, March/ April 2000, V.6, N.2:19

Doctors need to be able to monitor a patient's cardiovascular performance after the patient has been released from the hospital for surgery or other procedures. Monitoring is also important when the patient has begun a therapeutic drug regime. Unfortunately, the healthcare practitioner is not always available to follow the patient around. Currently, some patients employ “home healthcare” where a healthcare practitioner travels to the patient's home to provide the patient's care. Home healthcare can be expensive and is often not very cost-effective.

Also, the inability to monitor a patient on a frequent-interval basis can cause a doctor to choose to withhold certain critical, or potentially beneficial treatments, such as the administration of beta blocker type pharmaceuticals, because the doctor knows that the treatment protocol for some treatment regimes require heightened (very frequent) monitoring that is not available to patients being cared for in a home setting, or at some place away from a well equipped healthcare facility. The availability of hemodynamic monitoring systems has been limited by the lack of affordability, technical accuracy, and lack of easily obtainable hemodynamic parameters to guide the healthcare practitioner in designing and implementing a patient treatment strategy for the congestive heart failure patient. In particular, the limitations of existing hemodynamic monitoring systems have made it extremely difficult for a healthcare practitioner to optimize appropriate pharmacological therapy. The non-invasive hemodynamic technology of the present invention incorporates, and improves upon the Assignee's hemodynamic technology, which is described in more detail in Chio U.S. Pat. Nos. 4,880,013; 5,162,991; 5,836,884; and Chio and Brinton U.S. Pat. No. 5,879,307, the disclosures of which are incorporated herein by reference. The Applicant's technology allows reliable outpatient determinations of these hemodynamic parameters, potentially allowing the healthcare provider to make a tailored adjustment of the therapeutic regime being provided to patients with cardiac dysfunction.

The Assignee's Dynapulse® hemodynamic monitoring system⁵ can play a pivotal role in managing these patients without undermining the desirable objectives of cost containment, efficacy and safety. As heart failure rates continue to grow and further impact society, increased significance is placed on demonstrating positive outcomes in heart failure treatment techniques. It is believed that an effective patient management strategy can decrease hospitalizations for patients with costly chronic illness, including heart failure. ⁵Described in the Assignee's patents, and on www.dynapulse.com

Most previously reported heart failure management programs have been based solely upon telephone interventions, where subjective information is solicited from the patient and/or his family. The Applicants have developed a proprietary noninvasive approach to monitoring the hemodynamic parameters of such chronically ill patients. The Applicants believe that when such objective physiologic data is coupled with a patient's subjective input, the cost-effectiveness and efficiency of a “bad heart” monitoring and treatment program can be maximized.

### III. SUMMARY OF THE INVENTION

In accordance with the present invention, a method for remotely monitoring the cardiovascular condition of a patient, comprises using a data acquisition device at a patient site to non-invasively acquire cardiovascular condition information from a patient. The cardiovascular condition information includes a data stream of pulse pressure-related data. The cardiovascular condition information acquired by the data acquisition device is transmitted to a remote processor capable of performing data processing and data storage functions on the transmitted cardiovascular condition information. The cardiovascular condition information processed by the remote processor is transmitted to a data display device at a healthcare provider site remote from the data acquisition device for permitting the healthcare provider to use the cardiovascular condition information to monitor the cardiovascular condition of the patient.

One advantage of the present invention is that it enables a healthcare practitioner to monitor a patient's cardiovascular or other health conditions by monitoring the patient's health parameters, by acquiring cardiovascular data about the patient while the patient is away from a healthcare facility, subjecting the data to a sophisticated analysis and electronically conveying the data to a healthcare provider. The provider receiving the processed data can interpret the data to diagnose the patient's condition, to enable the healthcare provider to treat the patient more accurately, in a significantly more convenient and hence cost-effective environment than known presently.

Another advantage of the present invention is its ability to collect more data points about the patient's condition through increasing the frequency at which the patient's condition is tested/sampled. Increased sampling frequency facilitates establishing trend lines to chart the patient's progress, because the increased number of tests provides the treating physician with more data points than the once-a-week data point sets that one might receive by the patient coming into the healthcare provider's office once a week.

Yet another feature of the present invention is its ability to provide the potential to store the collected data on storage devices (e.g. servers, hard drives, storage disks, tapes, etc.) for large numbers (e.g. hundreds or thousands) of healthcare providers and health facilities, each of which such persons and facilities may individually serve large numbers (e.g., up to hundreds or thousands) of patients. An aspect of this feature is also to store data from millions of individual patients indirectly cared for by healthcare providers and health facilities. The present invention can permit new data belonging to a particular patient to be connected to the particular patient's stored data without confusion, thereby allowing an analysis of the data to be performed using the correct data for the intended patient.

Yet a further feature of the present invention is to analyze data received from a remote location in real-time (or close to real time) to extract useful health parameters such as those parameters discussed in the Chio '884 patent, from the transmitted data as soon as they are received in the server. This allows the healthcare provider to review the data without delay when necessary.

Another feature of the present invention is its ability to correctly retrieve the health parameters that result from analysis for each patient in a manner that ensures that the correct data is retrieved. This allows the delivery of the correct data for each patient that a particular healthcare provider wants to review.

Yet another feature of the present invention is that by lowering the cost of patient cardiovascular condition monitoring, the patient can afford to be monitored more often, and for a longer period of time before exhausting his/her monitoring budget that was set by his/her insurance company or other third party payor.

### IV. BRIEF DESCRIPTION OF THE DRAWINGS

FIG. 1 is a schematic representation of the components used in conjunction with the present invention;

FIG. 2 is a schematic representation of a small file format used for storing and for processing data obtained in accordance with the present invention;

FIG. 3 is a schematic representation of a large file format used for storing and for processing data obtained in accordance with the present invention;

FIG. 4 is a schematic representation of the hardware, software and communication elements used in the present invention; and

FIG. 5 is a schematic representation of a medium file format used for storing and/or processing data of the present invention.

### V. DETAILED DESCRIPTION

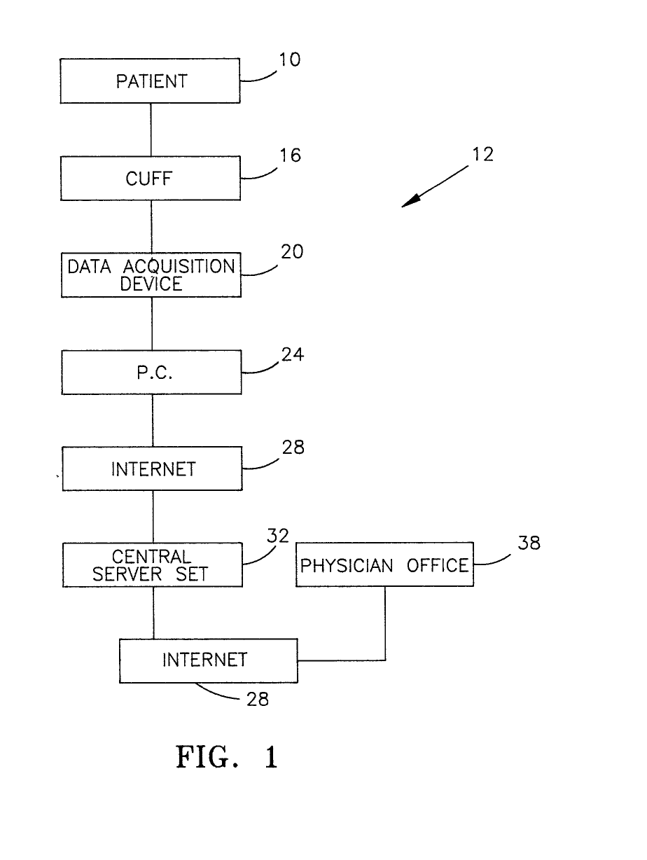

A flow of information starts at the patient 10 (FIG. 1), where he/she employs a data acquisition device 20 that records arterial wave forms for the patient when the patient's blood pressure is being taken. An example of such a device is the DYNAPULSE® Blood Pressure Monitoring device that is manufactured and sold by Pulse Metric®, Inc., of 11777 Sorrento Valley Drive, San Diego, Calif. USA 92121, the assignee of the present invention, and which is described in more detail in Chio U.S. Pat. No. 4,880,013, issued Nov. 14, 1989, and is discussed further within www.pulsemetric.com and/or www.dynapulse.com; and Chio U.S. Pat. Nos. 5,162,991 and 5,836,884 and Chio and Brinton U.S. Pat. No. 5,879,307. The Dynapulse® device and the disclosures of the Chio, and Chio and Brinton references are hereby incorporated herein by reference. The DYNAPULSE® device includes a conventional, inflatable blood pressure cuff 16 having an audio output tube that is coupled to a data acquisition device 20.

As explained in the Chio '013 patent, the data acquisition device 20 records and, to a limited extent, processes the incoming stream of data that is being acquired from the patient 10. Typically, the patient's data acquisition device 20 is capable of converting the analog data to digital waveform data. The data acquisition device includes software for processing the data to determine systolic and diastolic pressures and mean arterial pressure, and can also include a display to display such data to the patient 10. However, of the data obtained, the wave form data is generally the most important, and is the “raw” data that is used elsewhere in the process of the present invention, and which can be “mined” to determine other cardiovascular condition information.

The waveform information so received can be stored on a personal computer (PC) 24⁶, but alternately, can be stored in memory on the device 20 or elsewhere. The waveform data contains a copious amount of information about the patient's cardiovascular parameters. However in order to fully mine the patient data from the data acquisition device 20, the data of the arterial pressure waveform needs to be interpreted by an algorithm containing software system and/or human intervention. To do so, the waveform data is transmitted over a computer network, such as the Internet 28 to a central processing server 32 called an STE where the waveforms can be processed by software contained thereon, to determine, detect and derive additional cardiovascular parameters. The cardiovascular parameters so derived can then be transmitted to a healthcare provider 38, or to any other place of the healthcare provider's choosing 38, where the parameters can be used to monitor the patient's cardiovascular condition and/or be used to make additional diagnoses about the patient's condition. ⁶In view of converging and emerging technologies, such as personal data assistants, processor containing phones, game boxes, etc., the term “PC” is intended to be illustrative, and to incorporate devices known now, and which are invented in the future, that are capable of processing data and/or transmitting data over a computer network.

The patient's personal data acquisition device 20 not only records and derives traditional hemodynamic parameter numbers such as systolic pressure, diastolic blood pressure, mean arterial pressure and heart rate, but also records the continuous waveforms from the patient via the pressure cuff 16. The waveform can be used to determine systolic, mean arterial, and diastolic pressures as well as to determine information about arterial compliance and functional properties of the arteries. The waveform information also contains information about the flow of the patient's blood, cardiac contractility and various other hemodynamic parameters.

One of these other hemodynamic parameters is compliance. Basically, compliance is defined as the change in volume (of a blood vessel) divided by the change in pressure of the vessel. Compliance is used as a measure of the flexibility of blood vessels, and in particular the arteries in response to a change in pressure and relates to the mechanical properties of the arteries. Determining the arterial compliance of a patient can help a physician determine whether the patient suffers from arteriosclerosis, which is known also as hardening (stiffening) of the arteries. The present invention's ability to determine arterial compliance can be used to perform a semi-quantitative analysis of the degree to which the patient suffers from such ailments. Knowing something about a patient's arterial compliance allows the doctor to learn whether the patient may be at higher risk of cardiovascular disease. For additional information about the importance of determining compliance, and the methodology for determining compliance, see Chio U.S. Pat. No. 5,936,884, issued Nov. 17, 1998.

Knowledge of a patient's arterial compliance in conjunction with a knowledge of the blood flow through the patient's larger diameter arteries is also important because it provides information about a patient's cardiac output. Knowledge of a patient's cardiac output is useful for diagnosing the existence of possible heart valve problems.

Other parameters that may be derived from the waveform data include ejection fraction, cardiac output, and vascular flow velocity. Ejection fraction is the percentage of blood being ejected out of the heart divided by the potential volume of blood that the left ventricle of the heart can hold. A typical ejection fraction of a healthy person is between about 30 and 50 percent.

Still another parameter that can be derived is the left ventricle ejection time. This parameter can be used to determine whether the heart is having a difficult time ejecting blood, or if it is contracting too quickly. Also, the left ventricular (“LV”) contractility, the LV rate of change of pressure, the stroke volume index and cardiac output index can be derived from the waveform data. Through the present invention, these parameters can be monitored by a practitioner at his/her office or home, from information gathered by the patient's data acquisition system at the patient's site.

Once the waveforms containing all of the above-discussed data are acquired from the patient and fed into the patient's personal data acquisition system, the data acquisition system device 20 performs some processing of the waveforms within itself to determine (calculate) traditional hemodynamic parameters such as the patient's systolic, diastolic, and mean arterial pressures and the patient's heart rate. These calculated values, along with the waveforms are transmitted over the Internet to a central data center where the waveforms are further processed to generate hemodynamic parameters.

The data acquisition device 20, usually through the modem of the PC 24, is coupled via a cable, telephonic, or wireless connection to the Internet so that the device may send the waveform data to a data center, where the central server 32 resides. Critical patient information is preferably encrypted prior to transmission for patient privacy protection.

The central server (SET) 32 at the data center authenticates the user, and then accepts the remotely acquired patient data. The data is quickly processed by the SET and then stored in a relational database for facilitating searches and retrieval of the data by the patient's healthcare practitioner. Once in the database, a data management program, such as Microsoft SQL Server or Oracle, that resides on the SET 32 will mine the data in the data base to retrieve all of the patient's previously stored hemodynamic parameters. The recently acquired and processed parameters are then also stored in the database.

Next, the healthcare practitioner contacts the center, via the Internet and retrieves the report about his/her patient. When retrieving a patient's data, the healthcare provider can specify the particular measurement that he/she wants to retrieve and review. For example, she may decide to look at the patient's latest measurements. Alternately, she may review a series of measurements taken over a period of time to find a trend of the patient's condition.

Other embodiments exist such as one where the data is automatically delivered to a healthcare provider through e-mail or other client-end program. Also, a healthcare provider could simply be notified by e-mail that the center has new data about his patient, thereby providing the provider with an alarm and signal that alerts him automatically about the presence of additional data.

Preferably, the patient is able to restrict access and direct data to the specific database so that the patient may specify which healthcare providers are given access. Additionally, the patient's data is kept on the center's server so that the data can be seen by the patient's subsequent healthcare provider such as when the patient requires the services of a cardiologist or cardiovascular surgeon, other than his/her primary care physician.

After reviewing the data, the physician can then contact the patient by telephone or by e-mail to discuss the need for treatment, or to request that the patient take another set of measurements.

The ultimate diagnosis of the patient is made by the healthcare professional. However, the system also has the ability to recognize anomalies and to send an alert to either the patient or healthcare provider if an anomaly is detected. A healthcare provider can specify the parameters of normal operation so that an alert can be sent to the healthcare provider or patient when the patient's data deviates from the range of parameters set by the responsible provider. The alert can be one that alerts the healthcare provider to an adverse (or unexpectedly beneficial) change in the patient's condition; or can be one that alerts the patient to an anomaly in the data collected, thus suggesting to the patient that she re-perform the test upon herself and submit a second set of data. Such anomalies can occur if there is a significant change in the patient's condition or if the patient performs the pressure test incorrectly.

The data acquisition device 20 may be configured in one of several different ways. Some data acquisition devices are connected to a personal computer 24 so that the computer can perform some data processing functions, although such a device 20 should still contain sufficient memory and processing capability to process and store some information in the device 20. Some data acquisition devices are configured to work with a manually inflatable pressure cuff, whereas others are configured to include a pressurization and valve system to automatically inflate and deflate pressure cuffs. The advantage of storing the data within the data acquisition device 20 is that the device itself becomes ambulatory, and is not tied to a particular computer, thus permitting the data to be transported with the device.

Once the data is collected from the patient by the data acquisition device 20, it must be transferred via the Internet 28 to the SET 32. The data can be sent after each collection by the patient or, alternately, a series of collections can be taken and then transmitted simultaneously.

The data on the patient's data acquisition device 20 may be uploaded to the patient's PC 24. The PC 24 processes the collected patient data to convert the data into a file format that is readable by the central server (SET) 32. However, the file conversion could alternately be done at the central server (SET) 32. After the patient collects the desired amount of data (e.g. one reading or a series of readings) on the PC 24, the patient connects the PC 24 to the Internet 28, and the client program uploads the data via the Internet 28, to the central server 32.

The patient can keep a local copy of all the data on the long term memory (e.g. hard drive) of his/her PC, and each time that the PC communicates with the central server 32, the server 32 will note which data runs are new and will only download the new data runs, as the old data runs should already be stored on the central server 32.

Once the data is uploaded to the central server (SET) 32, it is stored with other data from the patient. The waveform data is stored along with other files that identify the patient's name, ID number, and background information. Either the patient or the healthcare provider may generate the commentary file and add comments to the file to create a commentary containing history of the patient.

Other embodiments exist where all the data is stored in a single file. In a single file embodiment, certain blocks of the single file will perform the functions of each of the individual identity, commentary, and other files.

When the waveform data is taken from the patient and transmitted to the server, (not before) the data may be filtered to remove noise or other unneeded parts of the waveform data. Multiple filters allow the waveform to be stored in multiple bands. Specifically, the waveform data can be recorded after passing it through a high pass, a low pass, and a band pass filter. These filters allow one to collect high frequency, low frequency and mid-frequency range data. All of the bands are placed into the waveform file of the data acquisition device 20 or personal computer 24 to be transferred to the central server (SET) 32.

Also, a master list file records the identities of all the different patients who have data stored on the remote PC. The master list file acts as a table of contents for the rest of the files on the remote personal computer 24 and/or data acquisition device 20. The master file list is especially useful in group use situations such as nursing homes, convalescent centers, and traveling home-healthcare worker situations, where a single remote personal computer 24 is being used to serve a plurality of patients.

The data acquisition software that is placed on the remote PC 24 is designed to access the master list file when the software is started up, and to present a list of the patients being monitored by that device 20/PC 24. This software permits the person running the test, (such as the health practitioner and/or patient,) to choose the appropriate patient file into which to place the soon-to-be-collected data.

The data block that is contained with the central server 32 may have multiple measurements contained within one block. Therefore, trend data can be ascertained from one or multiple data blocks for a review and/or processing of the multiple measurements.

The data can be placed in file form on the data acquisition device 20 or on the remote PC 24. The data is typically kept in raw form while stored in the data acquisition device 20, and is then converted to the file format once it is uploaded to the PC 24 through the processing capabilities of the PC 24. Also, the data can then be additionally converted into other file formats which are more conducive to being transferred over the Internet.

Also other embodiments exist wherein the data is transferred directly from a communications device (e.g. a modem) containing data acquisition device 20 across the Internet 28 to the central server 32, with no file formatting being performed by the user's data acquisition device 20 or PC 24.

Once the data is transferred to the central server 32 and analyzed in accordance with the teachings of the Chio patents disclosed above, and any other or newly developed procedures that may be useful, the healthcare provider may look at the reports that are generated from the processing of the patient data transferred to the server 32. Other embodiments can include the ability of the physician to access unprocessed waveforms and perform her own desired manipulation on the waveform data.

The master list file on the user's PC 24 need not be transferred to the server, since once the patient data is on the central server, the central server 32 database is able to generate a global patient list. The central server 32 may then take the uploaded data, and convert it into a form that is easily processed. Once the data is on the server 32 and in the desired format, the physician can look at any data set collected over any particular time interval to look for trends in the data.

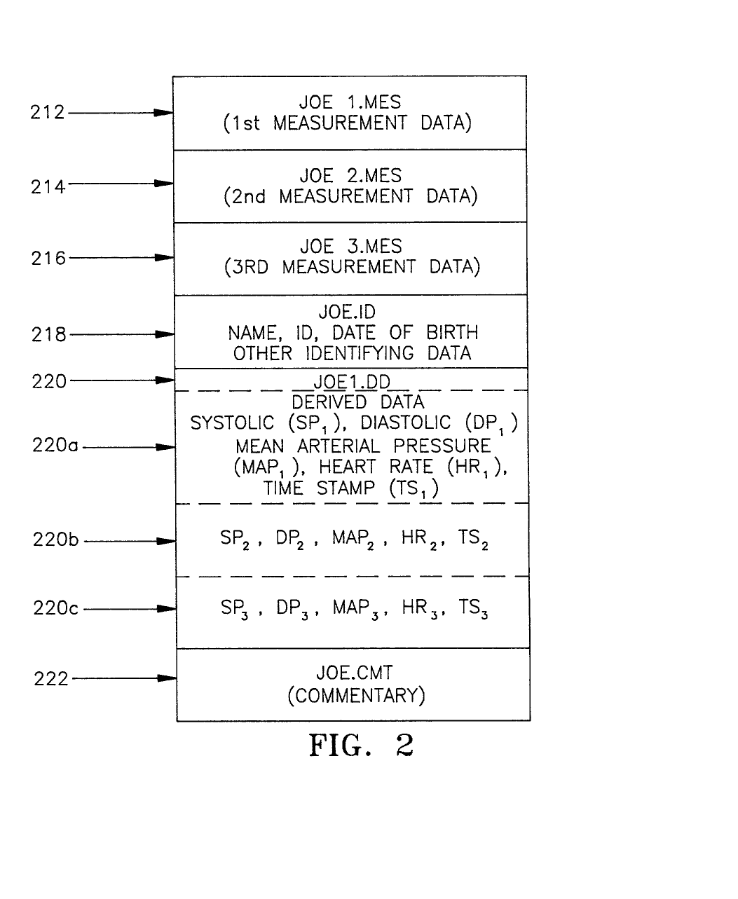

As noted earlier, several file formats are envisioned. The first is a small file format 210, that is represented schematically in FIG. 2. In the small file format 210, each patient has one group of data. If, for example, ten patients have data stored on the remote PC 24, there would exist ten groups of data. In this small file protocol, each group of data is associated with a patient on a different file name. The extension of the file designates what kind of file they have.

An exemplary file set 210 for a hypothetical patient, “Joe”, is shown in FIG. 2. Each patient has a collection of small files, the number of which is dependent upon the number of measurements stored for the particular patient. If the patient “Joe” has M number of measurements, he would have M plus three files. The file names are created according to his ID. Each measurement taken is used to create one measurement file (e.g. 212, 214, 216). In the hypothetical file set 210 for Joe, it is assumed that Joe has taken three measurements. The file set 210 includes three measurement files 212, 214, 216. These three measurement files contain the processed waveform data taken from a first measurement 212 performed on Joe, a second measurement 214 performed on Joe, and a third measurement 216 performed on Joe.

As stated above, the file also includes three non-measurement files 218, 220, 222. Non-measurement file 218 is the ID file 218 that includes various identifying indicia for Joe. Data within this ID file 218 includes such things as Joe's name, his patient ID number, his date of birth, and any other identifying data, such as cuff size, weight and height, that are deemed appropriate for inclusion. If desired, the ID file 218 can also include Joe's medical history, contact information, pharmaceutical regimes, etc. The second non-measurement file comprises a derived data file 220. The derived data file 220 includes a first block 220a, a second block 220b, and a third block 220c. The derived data in first data block 220a includes data derived from Joe's first measurement, that is, the waveform data contained within first measurement data file 212. The derived data includes Joe's derived systolic pressure (“SP”), derived diastolic pressure (“DP”), mean arterial pressure (“MAP”), and Joe's heart rate (“HR”). Additionally, the first derived data file block 220a includes a time stamp (TS) that contains information about the date and time on which the first measurement 212 was taken. For each measurement, the four parameters (SP, DP, MAP and Time Stamp) occupy one separate data block within the second non-measurement file 220.

Similarly, the second data block 220b within the derived data file 220 includes the derived systolic pressure (SP₂), diastolic pressure (DP₂), mean arterial pressure (MAP₂), heart rate (HR₂), and time stamp (TS₂) derived from, and corresponding to, the second measurement data contained in second measurement file 214. The third data block 220c contains the systolic pressure (SP₃), diastolic pressure (DP₃), mean arterial pressure (MAP₃), heart rate (HR₃), and a time stamp (TS₃) that were derived from Joe's third measurement, which waveform data is contained in the third measurement file 216. The final non-measurement data file (Joe.cmt) is a commentary file that contains comments that were entered either by the patient (Joe), or by Joe's healthcare provider.

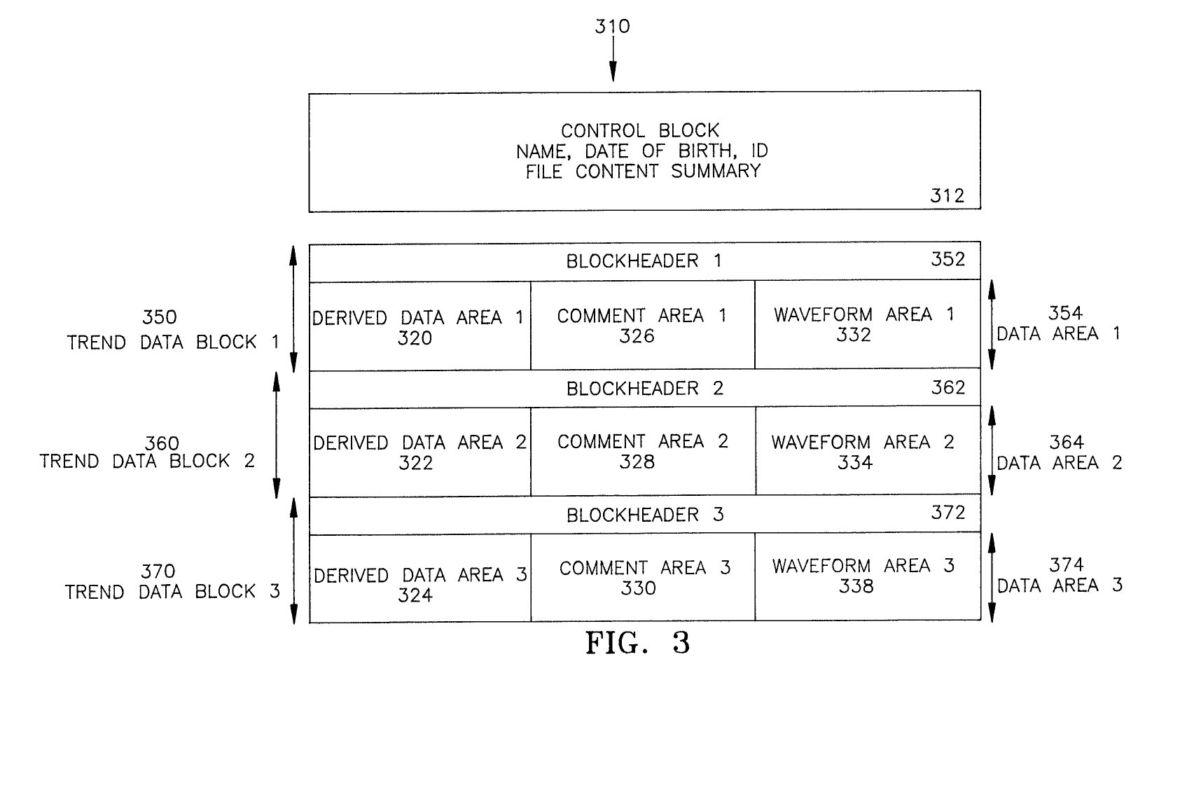

A second file format, herein referred to as “large file” format set 310 is shown in FIG. 3 for a hypothetical patient “Sally”. In the hypothetical file set 310 for Sally, it is assumed that Sally has taken three series of measurements. It should be noted that each series contains one or multiple measurements. In the large file format type file 310, all of Sally's data is contained within a single file 310. However, within large file 310 there is contained several different blocks of data. There is one control block 312 and three trend data blocks 350, 360 and 370. One trend data block exists for each of the three series of measurements shown in the exemplary file schematic of FIG. 3.

Within the control block 312 is contained a summary of the information contained within the file 310, and also contains patient identification data, such as Sally's name, date of birth, patient ID,. Also contained within file 310 is measurement data stored in trend data blocks.

The trend data block for each series of measurements includes one block header 352, 362, 372 and three data areas 320, 326, 332; 322, 328, 334; 324, 330, 338. The block headers 352, 362, 372 contain summaries of the data contained in the data areas 320-338, such as the number of measurements in the block, and data offset of each measurement within the block. For example, for the first measurement series, there is a “wave form” area 332 of data that includes the measurement information, primarily comprising waveform data for each measurement in the series; a derived data area 320 that contains data derived from the first series of measurements, such as systolic pressure, diastolic pressure, mean arterial pressure, heart rate, and a time stamp to indicate the time and date upon which each of the measurements in the series was taken. The final data area includes a comment data area 326, for storing one comment for each measurement in the series. Similarly, the second series of measurements includes a block header and three data areas, 322, 328, 324; the third series of measurements comprises a third block header and three data areas 324, 330, 336.

Although the large file format of FIG. 3 is shown as having three trend blocks 350, 360, 370 wherein one trend block is assigned for each of the three measurement series, the trend blocks may be assigned to be series independent, and characteristic specific. For example, trend block 350 may contain trend data from the first, second and third measurement series relating to derived systolic pressures taken on each of the measurements of all three measurement series. Similarly, the second trend block 360 may comprise the trend of diastolic pressures and/or mean arterial pressures taken from all three measurement series; and the third trend block 370 may, for example, contain heart rate data taken from all the three measurement series.

As such, one could use the data information from trend block one, to look at the trend of Sally's derived systolic, diastolic and mean pulse pressure or heart rate taken over the first series of measurements taken on Sally.

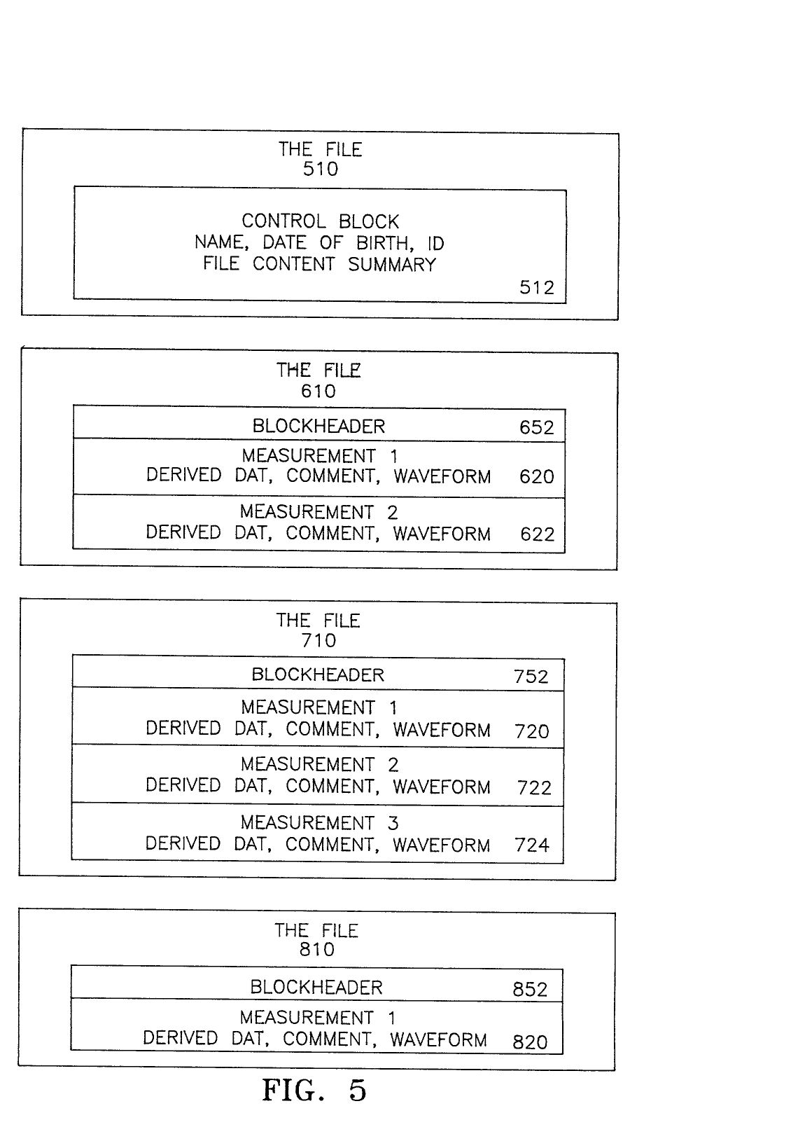

A third file format, herein referred to as a “medium file” format consisting of more than one file, 510, 610, 710 and 810 is shown in FIG. 5 for a hypothetical patient, Fred. In the hypothetical file set 510, 610, 710 and 810 for Fred, it is assumed that Fred has taken three series of measurements, containing 2, 3 and 1 measurements in the three series respectively. In the medium file-type, all of Fred's status is contained within a single file 510. However, the trend data is separated out into files 610, 710 and 810 for ease of access.

Similar to large file 310, medium file format 510 contains one control block 512. Unlike file 310, each single series of measurements is contained in one file, 610, 710 or 810. The structure of 610, 710 and 810 are identical, except that they may contain different numbers of measurement within the series each represents.

The following description uses 610 as an example, and this description also applies to files 710 and 810. For example, the block header 652 contains summaries of the data contained in the measurement data 620, 622, representing the first series of measurements. Similarly, the block header 752 contains information contained within the measurements 720, 722, 724 of the second series of measurements data block 516; and block header 852 contains a summary of data from the measurement 820 of the third series of measurements.

Similar to the large file format 310, for each measurement the derived data contains systolic pressure, diastolic pressure, mean-arterial pressure and heart rate data. The comment data includes any comments that are inserted by any of the healthcare provider, the patient or the processor and the waveform contains the waveform data collected from the device.

Unlike the large file format 310, where the derived data from all measurements in a series are grouped into one data area; all comments from all measurements in the series into another data area; and all waveform data into yet another block, the medium file format 610 groups the derived data, comment and waveform of each measurement of a series into one block.

The primary difference between the medium format file 510, 610, 710, 810 and the large format file 310 (FIG. 3), is the placement of trend data within its own file or sub-file. The placement within 610, 710, 810 facilitates data management in the server for the purpose of trend data extraction, storage and presentation. By applying the hemodynamic analysis of Chio '884 patent to the waveform data, hemodynamic trend data extracted can include cardiac parameters such as cardiac output, ejection fraction, contractility, stroke volume, systemic vascular parameters such as compliance and resistance, and brachial arterial parameters such as compliance, distensibility and resistence. As discussed above in connection with other embodiments, trend data is very useful to a healthcare practitioner, as it gives the healthcare practitioner data from the patient over a period of time. This data helps the practitioner better diagnose the patient's condition, as such trend data tends to reduce the importance of a anomalies, such as blood pressure spikes, that may occur in single measurements.

Additionally, the trend data can help the healthcare provider better determine whether the patient's condition is improving, or worsening. For example, if the trend data of the systolic, diastolic and mean arterial pressure, trend data shows that the diastolic blood pressure, systolic blood pressure, and mean arterial blood pressure are undergoing a rising trend, the healthcare practitioner will be alerted that the patient's condition is changing (and perhaps) worsening, thus providing a possible signal for the healthcare practitioner that additional intervention is necessary, or that current therapeutic and/or pharmaceutical regimes are not functioning properly. Similarly, a trending increase in the patient's cardiac output or ejection fraction can indicate that the heart of a patient is beginning to strengthen itself. Conversely, downwardly trending cardiac output and/or ejection fraction data would suggest that the patient's condition is worsening.

In practice, the number of hemodynamic parameters for which trend data will be extracted is determined by the selection of parameters that are being monitored and followed by the healthcare practitioner. Significantly, the blood pressure monitoring process of the present invention, and as disclosed in the earlier Chio patents, provides the potential to mine information from blood pressure data that was previously unable to be mined from such blood pressure data. As discussed in the Chio patents, information such as cardiac output, and ejection fraction previously required invasive measurements of the type which were not easily performed outside of a healthcare setting such as a doctor's office, clinic or hospital.

By placing the files in the medium file format, the information can generally be accessed more quickly, and more efficiently, than in the large file format 310, as the medium file format 510, 610, 710, 810 corroborate the use of the database in the central server 32 where extracted trend data are stored in a relational database for faster processing and retrieval on real-time demand by the user/healthcare practitioner.

When a healthcare provider wants to view a patient's information, the provider accesses the relational database at the central server 32 (FIG. 1). The server 32 is then searched. The provider is permitted to view the measurements of those patients(s) from whom she has permission.

Preferably, the central server 32 manipulates data when the data is in the medium file format. If the data is collected in either the large or small file format, the data is preferably converted to the medium file format at the remote PC before data transfer or within the central server 32. The converted medium file is named according to the ID of the patient, the time sequence of the data, and other helpful identifiers. Preferably, the ID is associated with the patient registered on the server 32. However, for security reasons, the data files should not actually contain the patient's name, but rather only identifying indicia. The patient's name is preferably disguised or omitted so that if an unauthorized person who somehow gains access to the file, and views the data file (notwithstanding security measurements), the unauthorized hacker will not be able to determine which patient the measurements came from, thus decreasing the risk of an unwanted invasion of the patient's privacy.

Based on the file name, the server 32 knows where to properly file that data. This creates an organizational scheme on the server 32. Further, the relational databases relate all the files to one another, and this established relationship aids in the searching of the data contained on the server 32. The server 32 may also open a particular file to gain further identification data and then use that data to electronically catalog the file.

Once a file such as file 510, 610 is logged into the database on the server 32, the file is flagged for analysis. Primarily, this analysis comprises the creation of trend data, and derived data, and the placement of such trend data and derived data in a presentation format that will be useful and understandable to the healthcare practitioner. An example of such a presentation format is a table showing the patient's various derived data figures, and a graph showing the patient's trend data over a particular time interval, such as a graph of the patient's systolic pressure taken over a series of ten measurements.

Additionally, as discussed above, the waveform information acquired from a patient can be mined and analyzed to determine other cardiac parameters such as cardiac output, LV Left Ventricular contractility, ejection fraction and arterial compliance. This type of cardiac parameter information can also be calculated and placed in an appropriate presentation format.

To perform such an analysis, an analysis program on the server 32 is run, which accesses all the records and files and pulls the relevant information from the database to perform the analysis. Once the analysis is completed, the program stores the resulting data in another table or in multiple tables in the database.

After the analysis is completed, the processed data is ready for the healthcare provider to review at whatever time the provider desires to log on to the central server 32 and retrieve the data. However, in cases where certain specific graphs and other illustrations of the patient's data exist that require large amounts of storage space on the central server 32, or otherwise require large amounts of processing time, the analysis program can be configured to perform the analysis or generate the table and/or graph on an “on-demand” basis only when so requested by the healthcare provider. Configuring the analysis program in this manner helps to save storage space and processing utilization of the server 32, as such processing capacity and storage space is only consumed if and when desired by the healthcare provider.

Also, if the data that is received from the patient is bad data due to the incorrect performance of the test by the patient, the healthcare provider can, by viewing the data (or by the use of an anomaly alarm generated by the server), detect the incorrectness and then contact the patient to remedy the data problems by, for example instructing the patient to re-perform the test. Additionally, the anomaly generator can be employed to send an alarm to the healthcare provider if the test results signal that the patient's results either fall outside of desired parameters, or indicate an unexpected deterioration in the patient's condition. Further, once the healthcare provider retrieves the data, she may download, copy, print, and export the file into another format or save the information into her own electronic or paper patient record system.

Placing the data within the server 32 in a medium file format, e.g. 510, 610, provides benefits over its placement in either the small format 210 or the large format 310. The small file format 210 suffers the disadvantage of creating many small files, one or more of which could be misplaced. The large file format 310 suffers the disadvantage of not being conducive to updating the information because each time that new data is to be uploaded to the server 32, the entire file (including old data that is already contained within the server) must be uploaded into the random access memory of the server and/or into the physician's remote computer that is being used to view the patient's data.

The use of the medium format 510, 610 has the advantage of reducing the impact of errors in transmission. If an error in transmission is made to data in the medium format, only one file or trend would be corrupted and only that file would need to be re-sent. Additionally, the medium file format 510,610 has the advantage of the file being able to be opened quicker than a file in the large file format 310, while still having a more significant amount of data contained within a single file than with a file in the small file format 210.

The server 32 creates the file names for the various patient files. Preferably, the file names are created in such a manner so as to allow a server administrator to know what type of a file a particular file is by the file extension, which extension also indicates to the administrator where the file should be stored within the database of the server. In case of a file mis-placement, the file name will tell the administrator where the file belongs and where its proper placement resides. Additionally, the data files may be stored in a compressed mode so as to take up less of the server's storage space.

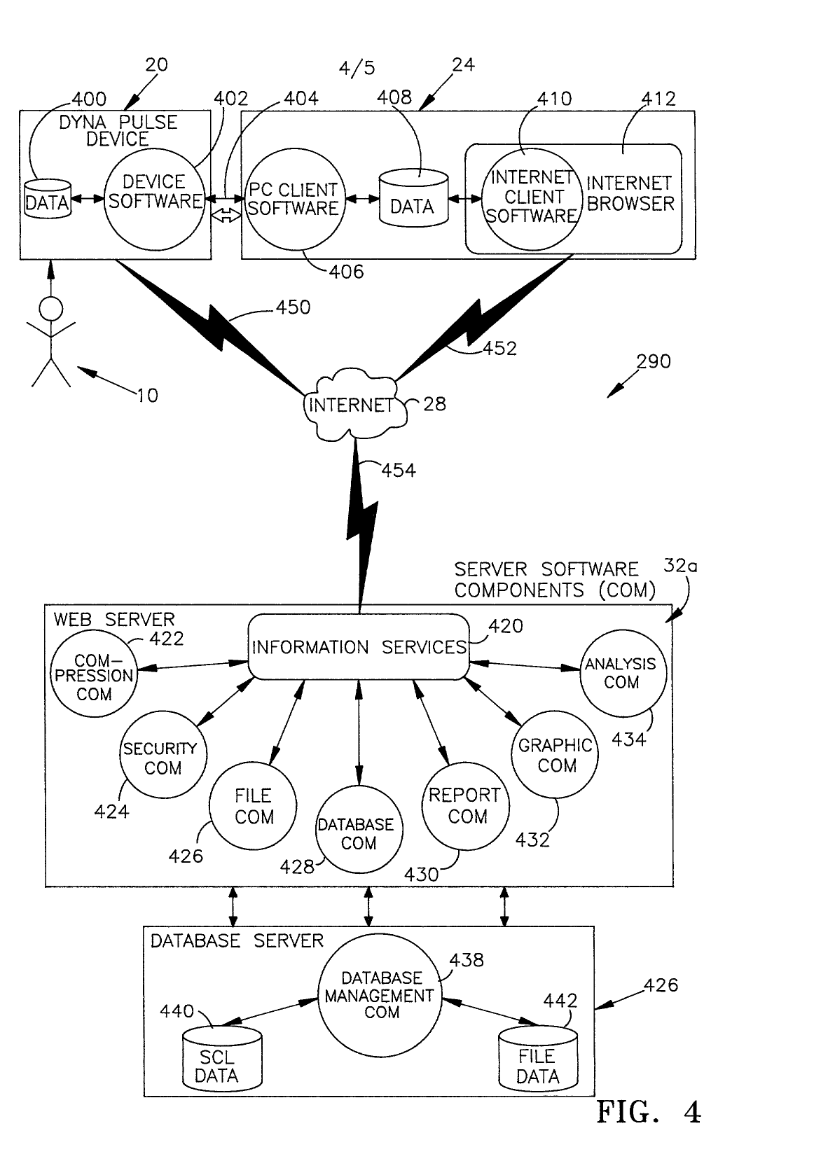

As such, the intelligence of this information management system is composed of a variety of, and plurality of software components running on various hardware devices, including the patient's data acquisition device (e.g. his DynaPulse device), the patient's personal computer, the several intermediary web servers, terminating at the web server used by the data processing center, and data processing center's database server. These components form the core of the information management system and are configured to, and equipped with software to interact with one another to carry out all the functions described above. FIG. 4 depicts an example of such a software/hardware system.

Turning now to FIG. 4, a highly preferred embodiment of the hemodynamic analysis device and method of the present invention is represented schematically. The hemodynamic analysis device 290 of FIG. 4 includes several major “types” or “categories” of components, wherein each major type includes several different individual components. The major component types include physical devices, such as the data acquisition device 20, and the remote PC 24, that are shown as sharp cornered rectangles; and an Internet operating system component, such as an information server 420 shown in round cornered rectangles. Software module components, such as the device software 402 that operates the DynaPulse® data acquisition device 20 are shown as circles; and on data storage devices, such as the data storage chip 400 contained within the DynaPulse® acquisition device 20, and the hard drive 408 of the remote PC 24 are illustrated as cylinders. On site, or intra-device data connections, such as data connection 404 between the data acquisition device 20 and the remote PC 24 are shown as arrow-headed lines, and a lightening blast, such as links 450, 452 and 454 are used to denote remote communication links.

The data acquisition device 20 includes a data storage means 400, that can comprise a memory chip, or alternately can comprise a hard disk, floppy disk or CD Rom drive. The data acquisition device 20 also includes software 402 for operating the device 20, and manipulating the storage and processing of data acquired by the data acquisition device 20. The data acquisition device 20 preferably includes a wired connection 404, or other communication link, such as an infrared or other radio transmission link to the PC 24, so that the data acquisition device 20 can communicate with the remote PC 24 located at the user's site. The remote (patient's) PC 24 also includes a data storage device 408, such as a hard drive; PC client software 406 that is used to communicate with the data acquisition device 20; and an internet browser software package 412 containing internet client software 410 that permits the remote PC 24 to communicate via the remote communication link 452 to the internet. Alternately, the data acquisition device 20 can be equipped with Internet browser and Internet client software (not shown) to permit the data acquisition device 20 to communicate through a remote communication link 450 to the internet 28.

As will be appreciated, the Internet comprises a global network of linked computers that permits the user to gain access to information on remote computers, and also transmit messages to remote computers. The Internet also, through a remote connection link enables communication to a web server 32a that is under the control of the analysis center provider. The web server includes information service software 420 that permits the web server 32a to communicate with the Internet 28, and ultimately to either the data acquisition device 20, or the remote PC 24.

The web server 32a contains a plurality of various software modules or components that permits the web server 32a to perform an analysis of the data it receives, and process the data stream of information that it receives from the user using the DynaPulse® device 20, and to communicate such analyzed information to a healthcare provider (not shown) having his own PC or other internet accessing appliance at the healthcare provider's site. Due to the great advances being made in information transfer technologies, it will be appreciated that the “Internet appliance” can comprise such diverse devices as a cellular telephone, a WebTV, a personal computer, or a personal digital assistant, or for that matter, any other internet accessing device that may hereafter be developed. In this respect, the particular form that the Internet accessing device takes is not nearly as important as the ability of the device, whatever its form, to permit the user to gain access to the Internet, to receive data therefrom, and transmit data there across.

As stated above, the web server 32a includes a plurality of software components. Among the software components illustrated in FIG. 4 are a compression module 422 that permits the data to be transferred over the Internet, and received from the Internet be to compressed and decompressed as necessary. A security module 424 is provided for encrypting necessary data to help protect patient privacy. A file component 426 is provided for organizing patient data, that is both received from the patient, and which is transmitted to either a patient or healthcare provider into a form that makes the data more easily processable by a database server 32b.

A database component 428 is provided for creating and providing a relational database that patient information to be stored and retrieved along with any other information reasonably necessary to permit the hemodynamic analysis system to perform its necessary functions.

Report module 430 is provided for generating necessary reports that relate to the operation of the web server 32a, and also generate reports relating to a particular patient's condition, and/or reports relating to the results of the analysis performed by the web server 32a. A graphics component software module 432 is provided for permitting the report to the patient to include graphical elements, to make the report easier to understand.

Finally, and most importantly, an analysis component 434 is provided that, as discussed above, performs an analysis of the patient waveform data transmitted into the web server 32a, to help analyze the raw patient waveform data to help display the physiological significance of this particular waveform data. As also discussed above, the particular analyses performed on the data are those various analyses discussed in the Chio patents and the Chio and Brinton patent. However, as new ways of analyzing the data are discovered, and new analysis procedures are invented, these new procedures and analysis techniques can be added to the analysis component, so that the analysis performed on the waveform data may be greater and more far-reaching than that analysis currently described by the Chio patents.

A database server 32b is in communication with the web server 32a, and can be a part of the web server 32a. The database server 32b contains the ability for storing both SQL data and file data. Additionally, the database server 32b includes a database management software component for managing the database created from the patient data. The database management software component 438, SQL data storage device 440, and file data storage device 442 are all in communication with each other. Although the SQL data storage device 440 and file data storage device 442 are shown as separate devices, it will be appreciated that they can comprise a part of the same storage device, such as part of the same hard drive, so long as the hard drive is capable of processing information quickly enough, and storing a sufficient amount of data to perform the functions required by the hemodynamic analysis system, at an acceptable rate.

Although the device has been described in connection with the presently perceived best mode of practicing the invention, it will be appreciated by those skilled in the art that variations, scope and spirit of the invention are significantly expanded beyond that which is shown and described in detail.

What is claimed is:

1. A method for remotely monitoring the cardiovascular condition of a patient, comprising:
using a data acquisition device at a patient site to non-invasively acquire cardiovascular condition information from a patient, the cardiovascular condition information including a data stream of pulse pressure-related data,
transmitting the cardiovascular condition information acquired by the data acquisition device to a remote processor capable of performing data processing and data storage functions on the transmitted cardiovascular condition information; and
transmitting the cardiovascular condition information processed by the remote processor to a data display device at a healthcare provider site remote from the data acquisition device for permitting the healthcare provider to use the cardiovascular condition information to monitor the cardiovascular condition of the patient.

2. The method of remotely monitoring cardiovascular condition of claim 1 wherein the step of providing wave form data includes the step of providing pulse pressure data for the patient taken over a period of time during which a blood pressure cuff exerted an elevated pressure on an appendage of the patient, and wherein data display device at a healthcare provider site comprises at least one of a display screen, a personal data assistant, a computer monitor, a phone screen and a printer.

3. The method of remotely monitoring cardiovascular condition of claim 1 wherein the step of providing pulse pressure data includes the step of providing pulse pressure data taken over a time interval during which the elevated pressure on the patient's appendage is varied between a supra-systolic elevated pressure and a sub-diastolic pressure.

4. The method of remotely monitoring cardiovascular condition of claim 1 wherein the step of transmitting the cardiovascular condition information includes the step of transmitting wave form blood pressure data for the patient taken over a time interval during which a blood pressure cuff exerts an elevated pressure on an appendage of the patient.

5. The method of remotely monitoring cardiovascular condition of claim 4 wherein the step of transmitting the cardiovascular condition information includes the step of transmitting patient indicia information to enable the remote processor to identify the patient from whom the cardiovascular condition information was being transferred.

6. The method of remotely monitoring cardiovascular condition of claim 1, wherein the step of using a data acquisition device includes the step of using the data acquisition device to process the non-invasively acquired information to determine at least one of the patient's systolic, diastolic and mean arterial blood pressures.

7. The method of remotely monitoring cardiovascular condition of claim 1 wherein the step of transmitting the cardiovascular condition information acquired by the data acquisition device includes the step of transmitting the systolic, diastolic and mean arterial blood pressure of the patient determined by the data acquisition device.

8. The method of remotely monitoring cardiovascular condition of claim 7, wherein the step of using a data acquisition device includes the step of using a data acquisition device comprising:
( a) a blood pressure cuff for exerting an elevated pressure on an appendage of a patient; and
(b) an information processor capable of being operatively coupled to the blood pressure cuff for receiving and performing processing operations on information received from the blood pressure cuff, the information processor being capable of being coupled to a display for displaying the systolic, diastolic and mean arterial pressure determined by the data acquisition device.

9. The method of remotely monitoring cardiovascular condition of claim 8, wherein the display is capable of displaying wave form data relating to the blood pressure of the patient as a function of at least one of time and pressure exerted by the blood pressure cuff.

10. The method of remotely monitoring cardiovascular condition of claim 1, further comprising the step of using the remote processor to process the cardiovascular condition information to create processed cardiovascular condition information, the processed cardiovascular condition information including at least one determined cardiovascular parameter.

11. The method of remotely monitoring cardiovascular condition of claim 10 wherein that at least one determined cardiovascular parameter is selected from the group consisting of systolic pressure, diastolic pressure, mean arterial pressure, heart rate, compliance, vascular resistance, ejection fraction, ejection time, ventricular contractility, cardiac output, vessel elasticity, stroke volume, ventricular rate of pressure change, bronchial arterial parameters, distensibility and arterial resistance.

12. The method of remotely monitoring cardiovascular condition of claim 1, further comprising the steps of establishing a patient identifier unique to a particular patient, establishing a healthcare provider identifier unique to a healthcare provider, and creating an association between the patient identifier and at least one healthcare provider identifier of a healthcare provider who provides care to the patient identified by the patient identifier, and wherein the step of transmitting the cardiovascular information to the healthcare provider includes the step of transmitting the cardiovascular information only to a data display device at a healthcare provider site having a healthcare provider identifier associated with the patient identifier.

13. The method of remotely monitoring cardiovascular condition of claim 1, further comprising the step of establishing a patient identifier unique to a particular patient, establishing a healthcare provider identifier unique to a particular healthcare provider, and establishing an association between a particular patient identifier and at least one particular healthcare provider identifier, for restricting access to cardiovascular information about a particular patient to those possessing an associated healthcare provider identifier.

14. The method of remotely monitoring cardiovascular condition of claim 13, wherein the step of establishing a patient identifier code includes the step of creating a patient identifier code devoid of any patient name or address information.

15. The method of claim 1 wherein:
the step of using a data acquisition device to non-invasively acquire cardiovascular condition information comprises the step of performing a series of cardiovascular data acquisition measurements to create a series of cardiovascular condition measurements, further comprising the steps of:
employing the remote processor to derive at least one cardiovascular parameter from each of the series of cardiovascular measurements, and correlating the derived cardiovascular parameters to create a trend report capable of being displayed at the healthcare provider site.

16. The method of claim 15 wherein the cardiovascular parameter is chosen from the group consisting of systolic pressure, diastolic pressure, mean arterial pressure, heat rate, compliance, vascular resistence, ejection fraction, ejection time, vessel elasticity, ventricular contractility, cardiac output, stroke volume, ventricular rate of pressure change, bronchial artery parameters, distensibility, and arterial resistance.

17. The method of claim 15 wherein the step of correlating the derived cardiovascular parameters includes the step of correlating the derived cardiovascular parameters temporally to create a temporally-based trend report.

18. The method of claim 17 wherein the step of creating a temporally-based trend report includes the step of preparing a graphic display for displaying the series of derived cardiovascular parameters as a function of time.

19. The method of claim 15 further comprising the step of creating a file within the remote processor for storing cardiovascular condition information received from a patient, and storing the trend data in a file format that permits the trend data report of a first cardiovascular parameter to be accessed separately from a trend data report of a second cardiovascular parameter.

20. Computer software for remotely monitoring the cardiovascular condition of a patient, the computer software being capable of being installed in a processor remote from each of a patient and a healthcare provider site, the computer software comprising:
a data receiving function for acquiring a data stream of cardiovascular information acquired non-invasively from a patient,
a processing function for processing the cardiovascular information to derive at least one cardiovascular parameter from the data stream received from the patient;
a storage function for storing the processed information in a manner that will correlate the processed information to make it accessible based on a patient identifier, and
a transmitting function that is capable of transmitting the processed information to the remote healthcare provider site to permit the healthcare provider to monitor the cardiovascular condition of the patient.

21. The computer software of claim 20 wherein the processing function is capable of processing the information received to derive at least one cardiovascular parameter selected from the group consisting of systolic pressure, diastolic pressure, mean arterial pressure, heart rate, compliance, vascular resistance, ejection fraction, ventricular contractibility, stroke volume, cardiac output, ventricular rate of pressure change, brachial arterial parameters, distensibility and arterial resistance.

22. The computer software of claim 20, wherein the data receiving function is capable of acquiring a series of cardiovascular measurements, the processing function is capable of deriving at least one cardiovascular parameter from each of the series of cardiovascular measurements, and correlating the derived cardiovascular parameters to create a trend report capable of being transmitted to the healthcare provider site.

23. The computer software of claim 22 wherein the processor is capable of correlating the derived cardiovascular parameters temporally to create a temporally-based trend report.

24. The computer software of claim 23 wherein the storage function is capable of assembling the data from each similar cardiovascular parameter into a file, for facilitating the creation of a trend report for the particular cardiovascular parameter.
